(19) United States
(12) Patent Application Publication
(10) Pub. No.: US 2003/0017192 A1
(43) Pub. Date: Jan. 23, 2003

(54) Process for producing extended shelf-life ready-to-use milk compositions containing probiotics

(76) Inventors: Hanny Kanafani, Newburgh, IN (US); Lorna Mize, Newburgh, IN (US)

Correspondence Address: Nelson Mullins Riley & Scarborough, LLP, Keenan Building, Third Floor, 1330 Lady Street, Columbia, SC, 29201, US

(21) Appl. No.: 10/176,121

(22) Filed: Jun. 18, 2002

Related U.S. Application Data
(60) Provisional application No. 60/299,288, filed on Jun. 19, 2001.

(51) Int. Cl. A61K 47/00; C12N 1/20

(52) U.S. Cl. 424/439; 424/93.4; 424/93.45; 435/252.1

(57) ABSTRACT

A process for producing extended shelf-life ready-to-use milk compositions containing probiotics by ultrapasteurizing a milk composition, cooling the composition to about 20 to 30° C., and inoculating the composition with one or more aseptically prepared probiotic cultures. The resulting milk compositions are ready-to-use, have an extended shelf-life, and contain sufficient probiotics to be beneficial to the consumer, even after an extended shelf-life of more than 90 days. The resulting milk compositions are used to provide probiotics to consumers in an effort to improve the consumer's intestinal health.

### BACKGROUND OF THE INVENTION

The present application claims the benefit of U.S. Provisional Application Serial No. 60/299,288 filed Jun. 19, 2001, which is incorporated herein by reference thereto.

1. Field of the Invention

This invention relates generally to processes for producing milk compositions and particularly to processes for producing extended shelf-life ready-to-use milk compositions containing probiotics.

2. Description of the Prior Art

### Probiotics

Probiotics are reported to have various health benefits for consumers, e.g., inhibition of bacterial pathogens, reduction of colon cancer risk, stimulation of immune response, and reduction of serum cholesterol levels. While there are several ways to administer probiotics to consumers, one convenient way is to simply add probiotics to foods that would normally be consumed, e.g., milk and yogurt. However, to get the desired health benefits, the probiotics must be selected carefully and added to foods in sufficient amounts to ensure that the recommended dose of probiotics is consumed. Also, the foods must be processed and handled in a manner that maintains the viability of the probiotic microorganisms during the manufacturing process and the time such foods spend on the shelf waiting for sale and consumption. Unfortunately, many of the probiotics added to foods are killed during the manufacturing process or simply die while the product stands on the shelf for extended periods.

The probiotics used in milk compositions must be carefully selected to ensure that the probiotics are compatible with the milk composition. Several genera of probiotic bacteria useful with milk compositions are known, e.g., Lactobacillus and Bifidobacterium. Of these, bifidobacteria are arguably the most studied and most important. Bifidobacteria are the predominant bacteria in feces of breast-fed infants. The unique flora bacteriology of Bifidobacterium provides protection in breast-fed infants against enteral as well as systemic disorders caused by bacterial pathogens such as coliform and streptococci. Coliform and streptococci are common microflora that cause neonatal infections such as gastroenteritis. To combat these infections, bifidobacteria metabolize lactose to produce acetic and lactic acid and increase intestinal acidity. Increased intestinal acidity is believed to inhibit the growth of such pathogens and increase the resistance of breast-fed infants to neonatal diseases such as infective gastroenteritis.

However, the beneficial effects of bifidobacteria and other probiotics are possible only if the probiotics are viable and have an affinity that permits them to colonize the human intestine. To produce therapeutic benefits, the minimum suggested level of viable bifidobacterial cells in yogurt at the time of consumption is approximately 10⁶ colony forming units (“cfu”) per ml or gram of product. In Japan, the Fermented Milks and Lactic Acid Bacteria Beverages Association requires a minimum of 107 viable bifidobacteria cells per ml to be present in fresh dairy products that claim to contain bifidobacteria (Ishibashi, N. and S. Shimamura. 1993. Bifidobacteria: research and development in Japan. Food Technology 47: 126, 129-34). The Swiss Food Regulation and the International Standard of FIL/IDF require that dairy products containing bifidobacteria contain no less than 10⁶ cfu per ml or gram of product (Shin, H-S, Lee, J-H., Pestka, J. and Ustunol, Z. 2000. Growth and viability of commercial Bifidobacterium spp in skim milk containing oligosaccharides and inulin. J. Food Sci 65(5):884-887). Probiotic organisms often show poor viability in these products. Commercially available pasteurized milk products that contain live probiotic bacteria have a typical shelf-life of only about 2 weeks. Further, evaluation of the viability of bifidobacteria in commercial unfermented milks sold in the United States over a 18-day period shows a 71% reduction in bifidobacteria population by the product expiration date and a bifidobacteria count of just slightly over 10⁶ cfu per ml of product (Shin, H-S, Lee, J-H., Pestka, J. and Ustunol, Z. 2000. Viability of bifidobacteria in commercial dairy products during refrigerated storage. J. Food Protection 63(3):327-331). Several studies have been done to improve viability of probiotics in dairy products such as milk, yogurt, and cheese by controlling the species or strain of the probiotic used, oxygen, acidity, pH, temperature and the use of bifidogenic factors that will enhance viability of the probiotic. However, none have been successful in producing a product with an extended shelf life.

The recognized health benefits of Bifidobacterium and other probiotics in infants led to the development of infant formulas containing bifidobacteria that are currently marketed in some areas of the world, e.g., Europe and Asia. These infant formulas, however, are powdered forms of infant formula; they are not convenient ready-to-use milk compositions. There are several problems with these powdered infant formulas. They are often ineffective because the probiotics die during processing and storage and are therefore not available to the infant to produce the desired health benefit. Further, commercially available, sterilized, ready-to-use infant formulas are often preferred over powders because of the convenience and safety to the consumer. Improperly sterilized water used to reconstitute powdered infant formula may pose a health risk to the infant. Similarly, improper reconstitution of the powdered formula may result in either a lower or higher concentration of nutrients and probiotics than is beneficial to the infant.

### Milk Compositions

Commercial milk compositions are produced using pasteurization. Pasteurization requires that the milk be heated to specific temperatures for specific times. The pasteurization process kills all pathogens and most of the microorganisms responsible for spoilage. Milk produced according to this process has a refrigerated shelf-life for about 15 days. Commercial milk compositions containing probiotics can be produced by adding the probiotic to milk prior to pasteurization but the pasteurization process will kill many of the probiotic microorganisms and thus prevent the consumer from getting a sufficient dose of probiotics when the milk is consumed. Similarly, milk compositions containing probiotics can be produced by adding the probiotic to milk after pasteurization. For example, U.S. Pat. No. 5,902,575 discloses a commercial milk composition containing probiotics that was made by adding a probiotic mixture to 1% pasteurized and vitaminized low fat milk just before filling the milk into its container.

In the preferred process, commercial milk compositions containing probiotics are produced by adding the probiotic to milk after pasteurization. The probiotics have to compete with other microorganisms growing in the composition. Research shows that milk compositions containing probiotics such as Bifidobacterium spp and Lactobacillus spp have a typical shelf-life of about 20 days when stored at about 4° C. and that only about 30% of the probiotics remain viable at the end of the shelf-life (Shin, H-S, Lee, J-H., Pestka, J. and Ustunol, Z. 2000. Viability of bifidobacteria in commercial dairy products during refrigerated storage. J. Food Protection 63(3):327-331). Methods for increasing the shelf-life and improving viability have included looking for improved strains and adding various compounds such as ascorbic acid and growth factors to the composition. As a result of these limitations, current commercial milk compositions containing probiotics may not have the number of probiotic microorganisms needed to confer the desirable health benefits. Also, such compositions spoil in about 20 days; what is not sold and consumed has to be discarded as waste.

There is, therefore, a need for new and improved milk compositions that have an extended shelf-life, e.g., greater than 90 days, and that contain probiotics that remain viable during such extended period.

### SUMMARY OF THE INVENTION

It is, therefore, an object of the invention to provide a process for producing extended shelf-life ready-to-use milk compositions containing probiotics.

It is another object of the present invention to provide extended shelf-life ready-to-use milk compositions containing probiotics.

It is a further object of the invention to provide extended shelf-life ready-to-use milk compositions containing probiotics that remain viable during an extended shelf-life.

It is another object of the present invention to provide extended shelf-life ready-to-use milk compositions containing probiotics in amounts sufficient to supply the consumer with the recommended dose of viable probiotics.

It is another object of the present invention to provide extended shelf-life ready-to-use milk compositions containing probiotics in amounts sufficient to benefit the consumer's health.

These and other objects are achieved using a novel process wherein a milk composition is ultrapasteurized, cooled to about 20 to 30° C., and inoculated with a probiotic culture that has been prepared under aseptic conditions. The resulting milk compositions are ready-to-use, have an extended shelf-life, and contain sufficient probiotics to be beneficial to the consumer, surprisingly even after an extended shelf-life of more than 90 days. Probiotics are added to the composition in specific amounts so that the consumer gets a beneficial dose of probiotic. In a preferred embodiment, the process is used to produce infant formulas containing probiotics. Such infant formulas can be fed to infants to improve the microflora in the infants' intestines.

Other and further objects, features and advantages of the present invention will be readily apparent to those skilled in the art.

### DETAILED DESCRIPTION OF THE INVENTION

### Definitions

The term “extended shelf-life” as used herein means a period that a product can be stored without the quality falling below a certain minimum acceptable level. The minimum acceptable level for the milk compositions of the present invention requires that the compositions maintain substantially the same physical and chemical properties, e.g., taste, smell, color, viscosity, sedimentation, and the like, for at least 90 days and that the compositions contain viable probiotics in an amount of at least 80% of the inoculated amount when the compositions are stored under refrigerated conditions, i.e., about 4° C.

The term “ready-to-use” as used herein means a milk composition or an infant formula in the liquid form that is ready for consumption without the addition of other ingredients or additional water.

The term “probiotic” as used herein means a culture of live microorganisms that beneficially affects a man or animal by improving the properties of the indigenous microflora in the intestines.

The term “aseptic conditions” as used herein means an atmosphere essentially free of microorganisms and includes the filling of a commercially sterilized, cooled milk composition into pre-sterilized containers followed by aseptic hermetical sealing with a pre-sterilized closure in an atmosphere essentially free of microorganisms.

The term “pasteurization” as used herein means a process wherein a milk composition has been heated either to (1) 145° F. for 30 minutes, (2) 161° F. for 15 seconds, (3) 191° F. for 1 second, (4) 204° F. for 0.05 seconds, or (5) 212° F. for 0.01 seconds.

The term “ultrapasteurization” as used herein means a process wherein a milk composition has been heated to at least 280° F. for at least 2 seconds so as to produce a milk composition that has an extended shelf-life under refrigerated conditions.

The term “commercially sterilized” as used herein means the condition achieved (1) by the application of heat which renders the food free of: (a) microorganisms capable of reproducing in the food under normal non-refrigerated conditions of storage and distribution; and (b) viable microorganisms (including spores) of public health significance; or (2) by the control of water activity and the application of heat, which renders the food free of microorganisms capable of reproducing in the food under normal non-refrigerated conditions of storage and distribution.

The term “infant formula” as used herein means a composition that satisfies the nutrient requirements of an infant by being a substitute for human milk. In the United States, the contents of an infant formula is dictated by the federal regulations set forth at 21 CFR Sections 100, 106, and 107. These regulations define macronutrient, vitamin, mineral, and other ingredient levels in an effort to simulate the nutritional and other properties of human milk.

### The Invention

In one aspect, the present invention is a process for producing extended shelf-life ready-to-use milk compositions containing probiotics. The process comprises the steps of:

ultra-pasteurizing a milk composition;

cooling the ultra-pasteurized milk composition to a temperature of from about 20 to 30° C. while maintaining aseptic conditions;

preparing a probiotic culture selected from the group consisting of Bifidobacterium genera, Lactobacillus genera, and combinations thereof under aseptic conditions; and

inoculating the probiotic culture into the cooled ultrapasteurized milk composition under aseptic conditions in amounts sufficient to produce a milk composition having a probiotic concentration composition of at least 1×10⁸ probiotic microorganisms per milliliter.

The resulting milk composition containing the probiotics is placed in sterile containers and sealed with sterile closures under aseptic conditions. In a preferred embodiment, the cooled ultrapasteurized milk compositions are filled into in sterile containers, the probiotic is inoculated into the composition in the container under aseptic conditions, and the container is sealed with a sterile closure under aseptic conditions. In one embodiment, the containers are flushed under aseptic conditions with a sterile inert gas, typically nitrogen, to remove air (oxygen) from the container just before sealing. Removing the air prevents the death of many anaerobic microorganisms such as Bifidobacterium due to oxygen toxicity. If the air remains, oxygen toxicity can result in a significant loss in concentration of the probiotics during production and storage.

Milk compositions useful in the present invention are milks obtained from mammals such as humans, bovines, ovines, equines, and the like. Typical animals include cows, sheep, goats, buffaloes, camels, llamas, mares, and deer. The milk compositions of the present invention also include soy milk. As used herein, soy milk refers to a liquid made by grinding dehulled soy beans, mixing the ground beans with water, cooking the mixture, and recovering the dissolved soy milk from the beans. Such soy milk can be formed into a milk-like product that has a taste, texture, and appearance similar to animal milk. Similarly, the milk compositions can be whey hydrolysate-based milk compositions or casein hydrolysate-based milk compositions. The milk compositions can be from a single species or compositions made from combinations of milk from one or more species or soy, e.g., a mixture of human and cows milk or a mixture of soy and cows milk. In one preferred embodiment, the milk composition is selected from the group consisting of whole cow's milk, skim milk, lactose-free milk, soy-based milk, whey hydrolysate-based milk, casein hydrolysate-based milk, and mixtures thereof.

In a preferred embodiment, the milk composition is an infant formula. The infant formula may be ready-to-feed (ready-to-use), i.e., a formula that may be consumed without requiring additional compositional changes such as the addition of water, preferably sterile water, prior to consumption, or a reconstituted powdered infant formula made by mixing water with powdered formulas such as those available commercially from Mead Johnson & Company (Enfamil® Infant Formula) or Ross Laboratories (Similac®) Infant Formula).

Probiotics useful in the present invention are any probiotics compatible with typical milk compositions, including infant formulas. Preferably, the probiotics are selected from the group consisting of Bifidobacterium and Lactobacillus genera, e.g., Lactobacillus acidophilus and Bifidobacterium bifidum. Most preferably, the probiotics are selected from the group consisting of Bifidobacterium lactis spp.

The probiotic is inoculated into the ultrapasteurized milk composition in amounts sufficient to provide a dose of probiotic as recommended by health professionals for the particular probiotic being added to the composition. Generally, the probiotic will be added to the milk composition in amounts sufficient to produce a milk composition having a probiotic concentration of at least 1×10⁸ probiotic microorganisms per milliliter. Preferably, Bifidobacterium lactis spp are added to the composition is amounts sufficient to produce a concentration of at least 1×10⁸ probiotic microorganisms per milliliter.

The milk compositions of the present invention can contain one or more different probiotics or different types of probiotics.

Various methods known to skilled artisans can be used for aseptically preparing the probiotic cultures of the present invention. A preferred process comprises preparing a probiotic suspension by aseptically weighing the required amounts of microorganisms into a sterilized glass bottle, closing the bottle with a sterilized cap, sterilizing reverse-osmosis water in an autoclave at 121° C. for 20 minutes, mixing the sterile water aseptically into the glass bottle containing the probiotic, and capping the bottle with a sterile container. If desired, the probiotic can be dispersed by shaking. Similarly, methods known to skilled artisans can be used to inoculate the probiotics into the sterile cooled milk composition. Generally, the probiotics are simply added by injecting the required amounts of probiotic solution into the compositions under aseptic conditions using a sterile injection device such as a pipette or needle.

The milk compositions of the present invention have an extended shelf-life of at least 90 days, preferably at least 120 days. Indeed, regression analysis of the stability data for the present milk compositions show them to be stable for about 3000 days.

Oxygen scavengers can be added to the composition to prevent loss of viability of the probiotics. Ascorbic acid or any oxygen scavenger known in the food industry and compatible with the composition may be used. In a study conducted to improve viability of probiotic bacteria in yogurts, ascorbic acid level of 250 mg/kg product helped improve viability of Lactobacillus delbrueckii spp bulgaricus. (Dave, R. I. and Shah, N. P., 1997. Viability of yogurt and probiotic bacteria in yogurts made from commercial starter cultures. Int Dairy Journal 7:31-41). Similarly, any growth factors that help preserve the viability of the probiotics can be added to the composition. Any growth factor known in the food industry and compatible with the composition may be used, e.g., fructooligosaccharides, galactooligosaccharide, and inulin. The growth factor is added to the composition in amounts required to prevent loss of viability, typically in amounts of up to about 5% on a weight basis. The viability of Bifidobacterium spp in skim milk was greatest in the presence of fructooligosaccharide, glucooligosaccharide and inulin, in descending order, at a maximum level of 5% w/v (Shin, H. S., Lee, J. H., Pestka, J. and Ustunol, Z. 2000. Growth and viability of commercial Bifidobacterium spp in skim milk containing oligosaccharides and inulin. J Food Science 65(5):884-887).

Any container and closure capable of maintaining an aseptic environment during processing and storage can be used. Glass bottles, paper cartons, and plastic bottles are acceptable. Preferably, the containers have low oxygen permeability, are resistant to light transmission, and maintain their integrity during handling, e.g., glass bottles or aluminum-laminated packages.

In another aspect, the present invention provides extended shelf-life ready-to-use milk compositions containing probiotics produced according to the process described herein.

The milk compositions of the present invention are useful because they provide a convenient and economical method for delivering viable probiotics to a consumer in amounts required to benefit the consumer's health.

The invention having been generally described, the following examples are given as particular embodiments of the invention and to demonstrate the practice and advantages thereof. It is understood that the examples are given by way of illustration and are not intended to limit the specification or the claims to follow in any manner.

### EXAMPLE 1

### Preparation of Bulk, Ready-to-Use Infant Formula

Water (27,000 g) was heated to about 120° F. (110-130° F.). Liquid skim milk, liquid whey, lactose, mineral preparation (made by dissolving calcium phosphate, potassium citrate, sodium citrate and calcium chloride in 6,120 g of 150-160° F. water), mineral/trace mineral preparation (made by dissolving ferrous sulfate, sodium chloride and trace mineral premix in 120 g of 100-120° F. water) were added. An oil preparation was made by heating the fat blend, lecithin and vitamins ADEK₁ concentrate to 160-170° F. Mono- and diglycerides and carrageenan were then added to the oil preparation and mixed well. The milk/mineral preparation and oil preparation were then mixed together. This mixture was then heated to about 250° F. (245-255° F.) for 45 seconds using direct steam injection and cooled to about 160° F. (150-170° F.). The mixture was homogenized twice at 160° F. (150-170° F.) second stage pressure of 500 psig and 3000 psig total. The mixture was cooled to 40° F. (35-50° F.). The total solids was measured (it should be about 18%). The amount of water (qs water) to be added in order to get a 12.4% solids in the final preparation was calculated. The dry vitamin premix and nucleotide premix were dissolved into the qs water and then added to the mixture. The final preparation was stored in a covered tank. The result was a 120-liter batch of ready-to-use infant formula containing the ingredients shown in Table 1.

TABLE 1

| Ingredient | Amount (grams) | |
|---|---|---|
| Liquid whey | 6412.04 | grams |
| Fat blend | 4193.1 | |
| Liquid skim milk | 2294.81 | |
| Lactose | 2273.39 | |
| Potassium citrate | 93.56 | |
| Mono- and diglycerides | 86.80 | |
| Calcium phosphate | 50.22 | |
| Dry Vitamin Premix | 45.19 | |
| Lecithin concentrate | 44.33 | |
| Carrageenan | 33.91 | |
| Calcium chloride | 31.80 | |
| Sodium chloride | 16.92 | |
| Nucleotide premix | 8.35 | |
| Ascorbic acid | 8.11 | |
| Ferrous sulfate | 7.30 | |
| Sodium citrate | 5.46 | |
| Vitamin A, D, E, K₁ Concentrate | 3.89 | |
| Trace mineral premix | 3.65 | |
| Water, quantity sufficient to | 120 | liters |

### EXAMPLE 2

### Preparation of Bifidobacteria Liquid Suspension

The infant formula should contain no less than 1×10⁷ live bifidobacteria per ml throughout its shelf-life. To compensate the potential loss of viability during its shelf-life, an initial level of 1×10⁸ live bifidobacteria per ml of infant formula was targeted.

Bifidobacterium lactis was used in this study because it is the most documented strain and one of the most studied strains in probiotic research. Numerous clinical studies have been conducted on the use of Bifidobacterium lactis in infants and children.

A bifidobacteria suspension was prepared by aseptically placing 0.85 g of Bifidobacterium lactis BB12™ (Chr Hansen BioSystems, Milwaukee, Wis. ) that contained 1×10¹⁰ live Bifidobacterium lactis per gram into an empty 3-oz sterilized glass bottle and closing the bottle with a sterilized cap. The empty glass bottles and caps were sterilized by placing them in an autoclave at 250° F. for 20 minutes. Glass bottles containing 85 ml of reverse-osmosis water were sterilized in an autoclave at 250° F. for 20 minutes. A glass (85 ml) of this sterile water was emptied aseptically into the glass bottle containing the 0.85 g of Bifidobacterium lactis BB12, capped, and dispersed well by shaking. Three ml of this inoculate, when combined with 3-oz of infant formula (or other milk composition), will yield a product containing 1×10⁸ live bifidobacteria per ml.

### EXAMPLE 3

### Preparation of Ready-to-Use Infant Formula Containing Live Bifidobacteria

The bulk ready-to-use infant formula prepared according to Example 1 was ultra-pasteurized by indirect steam injection using a MicroThermics UHT/HTST Lab 25 unit (MicroThermics, Inc., Raleigh, N.C.).Prior to ultra-pasteurization, the unit was sterilized by maintaining steam in the inlet and outlet tubes at 270° F. for 30 minutes. The hood area was sanitized by cleaning all contact surfaces with 200 ppm chlorine sanitizer. The hood area was also equipped with an air filter. A positive air flow was also maintained to prevent outside air from coming into the sanitized hood area.

The infant formula was heated at 280° F. (138° C.) for 8 sec and cooled to 73-80° F. (23-270C). The infant formula was placed into sterilized 3-oz glass bottles and capped with sterilized closures. Glass bottles were selected as the packaging material because it is impermeable to gases like oxygen which may interfere with the viability study. Three ml of the bifidobacteria suspension prepared according to Example 2 were inoculated aseptically into the glass bottles and flushed with nitrogen (10 seconds at about 5 psi pressure) to remove oxygen in the headspace. The bottles were capped with sterilized closures. Precautions were astringently observed in order to avoid contamination of the infant formula.

To determine the sufficiency of the sterilization conditions for the bottles and caps, a microbiological (swab) test of the sterilized bottles and caps was conducted. The test yielded less than 10 cfu per bottle and cap total plate count.

It is important that the infant formula prior to inoculation of bifidobacteria does not contain other viable microorganisms that would compete with bifidobacteria. The presence of other microorganisms that would compete with bifidobacteria will shorten the shelf-life of the product. Bifidobacteria will hardly be competitive in the presence of other microorganisms and will, thus, be easily outnumbered. (Gomes, A, M. P. and Malcata, F. X., 1999. Bifidobacterium spp and Lactobacillus acidophilus: biological, biochemical, technological and therapeutical properties relevant for use as probiotics. Food Science and Technology 10:139-157).

To determine the efficacy of the ultra-pasteurization condition of 270° F. (138° C.) for 8 sec in killing microorganisms that may compete with bifidobacteria, product samples subjected to this heating condition were tested microbiologically. The samples yielded <10 cfu per ml total plate count.

To determine the presence of contaminants in the ultra-pasteurized infant formula inoculated with bifidobacteria that may have entered by way of filling, gassing and capping, negative control samples (ultra-pasteurized infant formula without bifidobacteria) were also prepared.

### EXAMPLE 4

### Storage and Monitoring of Viability

The experimental samples (with bifidobacteria) and control samples (without bifidobacteria) from Example 3 were stored under refrigerated conditions (4-7° C.) for 91 days. Two bottles each of the experimental and control samples were withdrawn each week. Bifidobacteria were enumerated as follows: The samples were plated in Lactobacilli MRS agar (Difco #0882-17-0). The inoculated plates were incubated at 98° F. (37° C.) for 72 hours in a Forma Scientific Model 1024 (Thermo Forma, Marietta, Ohio) chamber under anaerobic conditions (5% CO₂, 5% H₂ and 90% N₂). Duplicate bottles were also tested for developed acidity using a titrimetric method and pH using Orion Research digital pH meter (Orion Research, Cambridge, Mass.).The viability of the experimental and control samples are shown in Table 2.

TABLE 2
Viability (average of two log cfu/ml readings) of Bifidobacterium
lactis in ready-to-use infant formula during refrigerated storage

| | | Anaerobic Plate Count/ml |
|---|---|---|
| Incubation | Experimental Sample (with | of Control Samples |
| Time (Day) | Bifidobacterium lactis) | (average of two readings) |
| 0 | 7.83 | <10 |
| 7 | 8.02 | <10 |
| 10 | 7.82 | <10 |
| 14 | 8.03 | <10 |
| 22 | 8.24 | <10 |
| 29 | 7.93 | <10 |
| 35 | 8.11 | <10 |
| 42 | 8.00 | <10 |
| 49 | 8.05 | <10 |
| 56 | 8.16 | <10 |
| 63 | 7.90 | <10 |
| 79 | 7.70 | <10 |
| 91 | 7.95 | <10 |

Referring to Table 2, the controlled samples did not yield any anaerobic viable microorganisms based on the less than 10 cfu/ml readings on all samples throughout the storage period. This demonstrates the absence of contaminants during the preparation of the samples. The experimental samples maintained the targeted level of no less than 1×10⁷ live bifidobacteria per ml product throughout the 91-day storage period. It is highly probable that this level will be maintained way beyond 91 days. The viability values (average of two log cfu/ml readings) were plotted on the y-axis against time (days) on the x-axis. The curve yielded a linear regression equation of y=−0.0008x+8.0097. If the plot assumes a linear regression beyond 91 days of storage, it will take about 2,500 days for the product to reach a level of 1×10⁶ live bifidobacteria per ml product, the suggested minimum level of bifidobacteria of therapeutic benefit. Therefore, the data show that the present invention can significantly extend the shelf-life of milk compositions containing probiotics well beyond 100 days, most likely several hundred days. In contrast, commercially available non-fermented milk products that claim 1×10⁶ live probiotic/ml have a very limited shelf-life at refrigerated conditions of only about 21 days (Shin, H-S, Lee, J-H., Pestka, J. and Ustunol, Z. 2000a. Viability of bifidobacteria in commercial dairy products during refrigerated storage. Journal of Food Protection 63(3):327-331). These commercially available products are made by adding a probiotic culture mixture to pasteurized and vitaminized cow's milk just before filling into cartons (See U.S. Pat. No. 6,194,578).

The acidity and pH of the experimental and control samples monitored during the storage period are shown in Table 3.

TABLE 3
pH and developed acidity in ready-to-use infant formula with live B. lactis

| | Experimental Samples (with | | | |
|---|---|---|---|---|
| Incubation | Bifidobacterium lactis) | | Control Samples | |
| Time (days) | pH | Acidity (% w/w) | pH | Acidity (% w/w) |
| 0 | 6.14 | 0.01875 | 6.96 | 0.00485 |
| 7 | 6.21 | 0.00489 | 6.92 | 0.00040 |
| 10 | 6.92 | 0.00411 | 6.98 | 0.00185 |
| 14 | 6.58 | 0.00467 | 6.96 | 0.00277 |
| 22 | 6.85 | 0.00513 | 7.06 | 0.00279 |
| 29 | 6.61 | 0.00544 | 6.98 | 0.00323 |
| 35 | 6.39 | 0.00588 | 6.86 | 0.00274 |
| 42 | 6.54 | 0.00541 | 7.08 | 0.00272 |
| 49 | 6.43 | 0.00636 | 6.94 | 0.00316 |
| 56 | 6.46 | 0.00542 | 7.10 | 0.00366 |
| 63 | 6.47 | 0.00808 | 7.03 | 0.02697 |
| 79 | 6.34 | 0.00715 | 6.87 | 0.00364 |
| 91 | 6.45 | 0.01083 | 6.87 | 0.003200 |

Referring to Table 3, there were no significant changes in pH and acidity in both control and experimental samples during the storage period. Increases in acidity and subsequent decrease in pH would indicate metabolic activity of bifidobacteria. The acidity produced from the fermentation of lactose by the bifidobacteria would cause acid shock and kill the bifidobacteria. Therefore, the data show that the present invention can significantly extend the shelf-life of milk compositions containing probiotics. Making the milk composition sterile to kill microorganisms that would compete with the bifidobacteria prior to inoculation of bifidobacteria and refrigeration storage can maintain the viability of bifidobacteria and thereby significantly extend its shelf-life.

Obviously many modifications and variations of the present invention are possible in view of the above teachings. It is therefore to be understood that within the scope of the appended claims the invention may be practiced otherwise than as specifically described.

What is claimed is:

1. A process for producing an extended shelf-life ready-to-use milk composition containing one or more probiotics, the process comprising the steps of:
ultra-pasteurizing a milk composition;
cooling the ultrapasteurized milk composition under aseptic conditions;
preparing a probiotic culture; and
inoculating the probiotic culture into the cooled ultrapasteurized milk composition under aseptic conditions.

2. The process of claim 1 wherein the probiotic culture is selected from the group consisting of Bifidobacterium genera, Lactobacillus genera, and combinations thereof.

3. The process of claim 1 wherein more than one probiotic culture is prepared and inoculated into the cooler ultrapasteurized milk.

4. The process of claim 1 wherein the probiotic culture is inoculated in an amount sufficient to produce a milk composition having a probiotic concentration composition of at least 1×10⁸ probiotic microorganisms per milliliter.

5. The process of claim 1 wherein the probiotic culture is Bifidobacterium lactis spp.

6. The process of claim 1 wherein the ultrapasteurized milk composition is cooled to a temperature of from about 20° C. to about 30° C.

7. The process of claim 1 further comprising placing the ready-to-use milk composition containing probiotics in a sterile container and sealing the container with a sterile closure in an atmosphere essentially free of microorganisms.

8. The process of claim 7 wherein the container is flushed under aseptic conditions with a sterile inert gas before being sealed.

9. The process of claim 1 wherein the milk composition is selected from the group consisting of whole cow's milk, skim milk, lactose-free milk, soy-based milk, whey hydrolysate-based milk, casein hydrolysate-based milk, and mixtures thereof.

10. The process of claim 1 wherein the milk composition is an infant formula.

11. A process for producing an extended shelf-life ready-to-use milk composition containing one or more probiotics, comprising the steps of:
ultra-pasteurizing a milk composition;
cooling the ultrapasteurized milk composition to a temperature of from about 20° C. to about 30° C. while maintaining aseptic conditions;
preparing at least one probiotic culture selected from the group consisting of Bifidobacterium genera, Lactobacillus genera, and combinations thereof under aseptic conditions; and
inoculating the probiotic culture into the cooled ultrapasteurized milk composition under aseptic conditions in an amount sufficient to produce a milk composition having a probiotic concentration composition of at least 1×10⁸ probiotic microorganisms per milliliter.

12. The process of claim 11 wherein the probiotics are Bifidobacterium lactis spp.

13. The process of claim 11 further comprising placing the ready-to-use milk composition containing the probiotics in a sterile container and sealing the container with a sterile closure in an atmosphere essentially free of microorganisms.

14. The process of claim 13 wherein the container is flushed under aseptic conditions with a sterile inert gas before being sealed.

15. The process of claim 11 wherein the milk composition is selected from the group consisting of whole cow's milk, skim milk, lactose-free milk, soy-based milk, whey hydrolysate-based milk, casein hydrolysate-based milk, and mixtures thereof.

16. The process of claim 11 wherein the milk composition is an infant formula.

17. An extended shelf-life ready-to-use milk composition containing one or more probiotics, the milk composition produced according to the process of claim 1.

18. A process for producing an extended shelf-life ready-to-use infant formula containing one or more probiotics, the process comprising the steps of:
ultra-pasteurizing an infant formula;
cooling the ultrapasteurized infant formula to a temperature of from about 20° C. to about 30° C. while maintaining aseptic conditions;
preparing a probiotic culture selected from the group consisting of Bifidobacterium genera, Lactobacillus genera, and combinations thereof under aseptic conditions; and
inoculating the probiotic culture into the cooled ultrapasteurized infant formula under aseptic conditions in an amount sufficient to produce an infant formula having a probiotic concentration composition of at least 1×10⁸ probiotic microorganisms per milliliter.

19. The process of claim 18 wherein the infant formula in the ultra-pasteurizing step is a ready-to-use infant formula.

20. The process of claim 18 wherein more than one probiotic culture is prepared and inoculated into the cooler ultrapasteurized infant formula.

21. The process of claim 18 wherein the infant formula in the ultra-pasteurizing step is a reconstituted powdered infant formula.

22. The process of claim 18 wherein the probiotic is Bifidobacterium lactis spp.

23. The process of claim 18 further comprising placing the ready-to-use infant formula containing the probiotic in a sterile container and sealing the container with a sterile closure in an atmosphere essentially free of microorganisms.

24. The process of claim 23 wherein the container is flushed under aseptic conditions with a sterile inert gas before being sealed.

25. An extended shelf-life ready-to-use infant formula containing one or more probiotics, the infant formula produced according to the process of claim 18.
